(19) United States
(12) Patent Application Publication
(10) Pub. No.: US 2008/0207960 A1
(43) Pub. Date: Aug. 28, 2008

(54) Methods, compositions, and kits for post-hybridization processing of arrays

(76) Inventors: Eric Lin, Santa Clara, CA (US); Eric M. Leproust, San Jose, CA (US); Patrick J. Collins, San Francisco, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/713,377

(22) Filed: Feb. 28, 2007

(51) Int. Cl. C07H 21/04; C07C 19/08; C07F 7/02

(52) U.S. Cl. 570/123; 536/23.1; 556/400

(57) ABSTRACT

In some embodiments, the present disclosure relates to systems, compositions, and methods for treating a microarray. The compositions include a superwetting agent. The methods include contacting the microarray with an aqueous mixture including the superwetting agent after a hybridization step. Kits for carrying out the methods are also provided.

### BACKGROUND

Microarrays of DNA or RNA polynucleotides or oligonucleotides are state-of-the-art biological tools used in the investigation and evaluation of genes for analytical, diagnostic, and therapeutic purposes. Microarrays typically comprise a plurality of oligomers, synthesized or deposited on a glass support or substrate in an array pattern. The support-bound oligomers are called “probes”, which function to bind or hybridize with a sample of DNA or RNA material under test, called a “target” in hybridization experiments. Some investigators also use the reverse definition, referring to the surface-bound oligonucleotides as targets and the solution sample of nucleic acids as probes. Further, some investigators bind the target sample under test to the microarray substrate and put the oligomer probes in solution for hybridization. Either of the “target” or “probes” may be the one that is to be evaluated by the other. Thus, either one could be an unknown mixture of polynucleotides to be evaluated by binding with the other. All of these iterations are within the scope of the present disclosure. In use, the array surface is contacted with one or more targets under conditions that promote specific, high-affinity binding of the target to one or more of the probes. The targets are typically labeled with an optically detectable label, such as a fluorescent tag, so that the hybridized targets and probes are detectable with scanning equipment. DNA array technology offers the potential of using a multitude (e.g., hundreds of thousands) of different oligonucleotides to analyze changing mRNA populations.

### SUMMARY

In general terms, the disclosure relates to methods compositions, and systems for processing molecules associated with a surface. For example, after hybridizing a first oligonucleotide bound to a surface with a second unbound oligonucleotide, the hybridization product can be further subjected to post-hybridization treatment procedures, such as washing steps, soaking steps, and drying steps.

In some aspects, there are disclosed methods of treating the hybridization product (e.g., of a first oligonucleotide bound to a surface with a second unbound oligonucleotide), the methods comprising: contacting the surface with an aqueous mixture comprising a superwetting agent as disclosed herein.

In some aspects, there are disclosed buffers or buffer concentrates comprising: a superwetting agent and a buffer suitable for a washing procedure as described herein.

In some aspects, there are disclosed kits for carrying out the disclosed methods. In some embodiments, a kit can comprise a wash buffer as disclosed herein, and an array comprising one or more ligands immobilized on a surface of a solid support.

Additional advantages and novel features of the invention will be set forth in part in the description which follows, and in part will become apparent to those skilled in the art upon examination of the following, or may be learned by practice of the methods.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a flow diagram of some embodiments of hybridization and processing methods.

FIG. 2 illustrates a magnified view of a hybridized microarray after a washing step.

FIG. 3 illustrates a feature extraction line scan from FIG. 2.

FIG. 4 illustrates a magnified view of a hybridized microarray washed with buffer containing superwetting agent.

FIG. 5 illustrates a feature extraction line scan from FIG. 4.

FIG. 6 illustrates the distribution of features from a hybridized microarray.

FIG. 7 illustrates the distribution of features from a hybridized microarray subjected to treatment with superwetting agent.

FIG. 8 illustrates the distribution of background areas from a hybridized microarray.

FIG. 9 illustrates the distribution of background areas from a hybridized microarray subjected to treatment with superwetting agent.

### DESCRIPTION

Various embodiments of the present disclosure will be described in detail with reference to drawings, if any. Reference to various embodiments does not limit the scope of the disclosure, which is limited only by the scope of the claims attached hereto. Additionally, any examples set forth in this specification are not intended to be limiting and merely set forth some of the many possible embodiments for the claimed invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present disclosure, the preferred methods and materials are now described. Methods recited herein may be carried out in any order that is logically possible, in addition to a particular order disclosed.

All publications mentioned herein are incorporated herein by reference to disclose and describe the methods and materials in connection with which the publications are cited. The publications discussed herein are provided solely for their disclosure prior to the filing date of the present application. Nothing herein is to be construed as an admission that the present disclosure is not entitled to antedate such publication by virtue of prior invention. Further, the dates of publication provided may be different from the actual publication dates which may need to be independently confirmed.

The practice of the present disclosure will employ, unless otherwise indicated, conventional techniques of chemistry, biochemistry, molecular biology, and medicine, including diagnostics, which are within the skill of the art. Such techniques are explained fully in the literature.

The following definitions are provided for specific terms that are used in the following written description.

Definitions

The following terms are intended to have the following general meanings as they are used herein.

“Superwetting agent” refers to a compound or composition that has an ability to significantly enhance the distribution of the compound, the composition, or other formulation constituents, particularly target or probe compositions, onto and across a surface. “Surface” generally refers to the exterior boundary of an object or body, for example, a porous or non-porous solid or substrate, a liquid, a gas such as a bubble, and combinations thereof, such as a composite, foam, gel, and like formulations, and can include an interface between or within like or dissimilar materials. Thus, for example, a suitable wetting liquid will spread on a surface that has been treated with a suitable superwetting agent, for example, an array-liquid interface, an array-bubble interface, an array-superwetting agent interface, a superwetted array-liquid interface, a liquid-gas interface, a superwetted gas-liquid interface, and like surfaces or interfaces. A superwetted surface prevents, for example, a solid surface from being repellent to a wetting liquid. Superwetting agents of the present disclosure provide surface-wetting and surface tension lowering that are greater than compared to conventional wetting agents. A superwetting agent in the buffer formulations of the present disclosure can significantly lower the equilibrium (static) surface tension, dynamic surface tension, or both, of the resulting contacted surface. Thus, superwetting agents useful in the practice of the present disclosure can be identified and selected based on, for example, empirical or known chemical compatibilities, stabilities, surface tension properties, and like properties. Suitable superwetting agents can provide surface tensions of, for example, less than about 75 mN/m at 25° C. at a 0.1 weight percent concentration in water. Other suitable superwetting agents can provide surface tensions of, for example, less than about 65 mN/m at 25° C. at a 0.1 weight percent concentration in water. Still other suitable superwetting agents can provide surface tensions of, for example, less than about 55 mN/m at 25° C. at a 0.1 weight percent concentration in water. Yet still other suitable superwetting agents can provide surface tensions of, for example, less than about 45 mN/m at 25° C. at a 0.1 weight percent concentration in water. Still other suitable superwetting agents can provide surface tensions of, for example, less than about 35 mN/m at 25° C. at a 0.1 weight percent concentration in water. Yet still other suitable superwetting agents can provide surface tensions of, for example, less than about 25 mN/m at 25° C. at a 0.1 weight percent concentration in water. Yet still other suitable superwetting agents can provide surface tensions of, for example, less than about 20 mN/m at 25° C. at a 0.1 weight percent concentration in water. Generally, the lower the surface tension of a surface that has been contacted by a superwetting agent formulation of the present disclosure will produce superior results and performance reliability. Superwetting agents are known and are commercially available, as described herein. Surface-tension and interfacial-tension measurement methods and techniques are known to those skilled in the art.

“Surfactant” refers generally to any surface-active substance, such as detergent.

“Water soluble” refers to the dispersibility property of a substance in water and includes, for example, molecular dispersibility of the substance, particulate dispersibility of the substance in water, or both, at one or more temperatures.

“Mixture” refers to, for example, an aqueous solution, a dispersion, or a biphasic or multiphasic combination of disclosed ingredients or components, such as a superwetting agent, and other components of the buffered compositions. The buffer composition or mixtures of the present disclosure mixture are preferably aqueous solutions or dispersions.

“Nucleic acid” refers to a high molecular weight material that is a polynucleotide or an oligonucleotide of DNA or RNA.

“Polynucleotide” refers to a compound or composition that is a polymeric nucleotide or nucleic acid polymer. The polynucleotide may be a natural compound or a synthetic compound. In the context of an assay, the polynucleotide can have from about 20 to 5,000,000 or more nucleotides. The larger polynucleotides are generally found in the natural state. In an isolated state the polynucleotide can have about 30 to 50,000 or more nucleotides, usually about 100 to 20,000 nucleotides, more frequently 500 to 10,000 nucleotides. Isolation of a polynucleotide from the natural state can often result in fragmentation. The polynucleotides can include nucleic acids, and fragments thereof, from any source in purified or unpurified form including DNA, double-stranded or single-stranded (dsDNA and ssDNA), and RNA, including t-RNA, m-RNA, r-RNA, mitochondrial DNA and RNA, chloroplast DNA and RNA, DNA/RNA hybrids, or mixtures thereof, genes, chromosomes, plasmids, the genomes of biological materials such as microorganisms, for example, bacteria, yeasts, viruses, viroids, molds, fungi, plants, animals, humans, and like materials. The polynucleotide can be only a minor fraction of a complex mixture such as a biological sample. Also included are genes, such as hemoglobin gene for sickle-cell anemia, cystic fibrosis gene, oncogenes, cDNA, and like genetic materials.

Polynucleotides include analogs of naturally occurring polynucleotides in which one or more nucleotides are modified over naturally occurring nucleotides. Polynucleotides then, include compounds produced synthetically, for example, PNA as described in U.S. Pat. No. 5,948,902 and the references cited therein, all of which are incorporated herein by reference, which can hybridize in a sequence specific manner analogous to that of naturally occurring complementary polynucleotides.

The polynucleotide can be obtained from various biological materials by procedures well known in the art. The polynucleotide, where appropriate, may be cleaved to obtain a fragment that contains a target nucleotide sequence, for example, by shearing or by treatment with a restriction endonuclease or other site-specific chemical cleavage method.

In embodiments, the polynucleotide, or a cleaved fragment obtained from the polynucleotide, can be at least partially denatured or single-stranded or treated to render it denatured or single-stranded. Such treatments are known in the art and include, for instance, heat or alkali treatment, or enzymatic digestion of one strand. For example, double stranded DNA (dsDNA) can be heated at 90-100° C. for a period of about 1 to 10 minutes to produce denatured material, while RNA produced via transcription from a dsDNA template is already single-stranded.

“Oligonucleotide” refers generally to a two or more covalently bonded nucleotides, usually single-stranded, usually a synthetic polynucleotide but may be a naturally occurring polynucleotide. The oligonucleotide(s) are usually comprised of a sequence of at least 5 nucleotides, for example, 10 to thousands of nucleotides, such as 2,000 to about 10,000, preferably, 20 to 250 nucleotides, more preferably, 20 to 125 nucleotides, and desirably about 60 nucleotides in length.

Various techniques can be employed for preparing an oligonucleotide. Such oligonucleotides can be obtained by biological synthesis or by chemical synthesis. For short sequences, such as up to about 100 nucleotides, chemical synthesis can frequently be more economical as compared to the biological synthesis. In addition to economy, chemical synthesis provides a convenient way of incorporating low molecular weight compounds, modified bases, or both, during specific synthesis steps. Furthermore, chemical synthesis can be very flexible in the choice of length and region of target polynucleotides binding sequence. The oligonucleotide can be synthesized by standard methods such as those used in commercial automated nucleic acid synthesizers. Chemical synthesis of DNA on a suitably modified glass or resin can result in DNA covalently attached to the surface. This may offer advantages in washing and sample handling. For longer sequences standard replication methods employed in molecular biology can be used such as the use of M13 for single-stranded DNA as described in J. Messing (1983) Methods Enzymol., 101:20-78.

Other methods of oligonucleotide synthesis include phosphotriester and phosphodiester methods (Narang, et al., (1979) Meth. Enzymol., 68:90) and synthesis on a support (Beaucage, et al. (1981) Tetrahedron Letters, 22:1859-1862) as well as phosphoramidate techniques (Caruthers, M. H., et al., “Methods in Enzymology,” Vol. 154, pp. 287-314 (1988)) and others described in “Synthesis and Applications of DNA and RNA,” S. A. Narang, editor, Academic Press, New York, 1987, and the references contained therein. The chemical synthesis via a photolithographic method of spatially addressable arrays of oligonucleotides bound to glass surfaces is described by A. C. Pease, et al., Proc. Nat. Acad. Sci., USA, (1994) 91:5022-5026. Unless otherwise noted, terms oligonucleotide and polynucleotide are used interchangeably.

“Nucleotide” refers to the monomeric unit of nucleic acid polymers, i.e., DNA and RNA, whether obtained from a natural source or produced synthetically, which comprises a nitrogenous heterocyclic base, which is a derivative of a purine or pyrimidine, a pentose sugar, and a phosphate (or phosphoric acid). When the phosphate is removed, the monomeric unit that remains is a “nucleoside.” Thus, a nucleotide is a 5′-phosphate of the corresponding nucleoside. When the nitrogenous base is removed from the nucleotide, the monomeric unit that remains is a “phosphodiester.” “Nucleotide” can include its corresponding nucleoside and phosphodiester, and “oligonucleotide” can include its corresponding oligonucleoside and oligophosphodiester, unless indicated otherwise. The term “nucleotide” can include “modified nucleotide” that contains, for example, a modified base, sugar or phosphate group. The modified nucleotide can be produced by a chemical modification of a nucleotide either as part of the nucleic acid polymer or prior to the incorporation of the modified nucleotide into the nucleic acid polymer. For example, the methods mentioned above for the synthesis of an oligonucleotide may be employed. In another approach, a modified nucleotide can be produced by incorporating a modified nucleoside triphosphate into the polymer chain during an amplification reaction. Examples of modified nucleotides, by way of illustration and not limitation, include dideoxynucleotides, derivatives or analogs that are biotinylated, amine modified, alkylated, fluorophore-labeled, and like modifications, and can also include phosphorothioate, phosphite, ring atom modified derivatives, and like modifications.

“Target material” or “target” refers to a sequence of nucleotides to be identified, usually existing within a portion or all of a polynucleotide, usually a polynucleotide analyte. The identity of the target nucleotide sequence generally is known to an extent sufficient to allow preparation of various probe sequences hybridizable with the target material.

The target material usually contains from about 30 to 5,000 or more nucleotides, preferably 50 to 1,000 nucleotides. The target material is generally a fraction of a larger molecule or it may be substantially the entire molecule such as a polynucleotide as described above. The minimum number of nucleotides in the target material is selected to assure that the presence of a target polynucleotide in a sample is a specific indicator of the presence of polynucleotide in a sample. The maximum number of nucleotides in the target material is normally governed by several factors: the length of the polynucleotide from which it is derived, the tendency of such polynucleotide to be broken by shearing or other processes during isolation, the efficiency of any procedures required to prepare the sample for analysis, for example, transcription of a DNA template into RNA, and the efficiency of detection, amplification, or both, of the target nucleotide sequence, where appropriate.

“Nucleic acid probe” refers to an oligonucleotide or polynucleotide employed to bind to a portion of a polynucleotide such as another oligonucleotide or a target material. The design and preparation of the nucleic acid probes are generally dependent upon the sensitivity and specificity required, the sequence of the target material and, in certain cases, the biological significance of certain portions of the target material.

“Hybridization,” “hybridizing,” “binding” and like terms, in the context of nucleotide sequences, can be used interchangeably herein. The ability of two nucleotide sequences to hybridize with each other is based on the degree of complementarity of the two sequences, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency is achieved by elevating the temperature, increasing the ratio of co-solvents, lowering the salt concentration, and the like. Hybridization of complementary Watson/Crick base pairs of probes on the microarray and of the target material is generally preferred, but non-Watson/Crick base pairing during hybridization can also occur.

Conventional hybridization solutions and processes for hybridization are described in J. Sambrook, Molecular Cloning: A Laboratory Manual, (supra), incorporated herein by reference. In some embodiments, conditions for hybridization include (1) high ionic strength solution, (2) at a controlled temperature, and (3) in the presence of carrier DNA and surfactants and chelators of divalent cations, all of which are known in the art.

“Complementary” refers to two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence, to form Watson/Crick base pairs. RNA sequences can also include complementary G=U or U=G base pairs. Non-standard or non-Watson/Crick base pairing is also possible with nucleotide complements, for instance, the sequences may be parallel to each other and complementary A=C or G=U base pairs in RNA sequences or complementary G=T or A=C base pairs in DNA sequences can occur, although not preferred.

A chemical “array”, unless a contrary intention appears, includes any one, two or three-dimensional arrangement of addressable regions bearing a particular chemical moiety or moieties (for example, biopolymers such as polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties can be covalently or non-covalently bound to the surface in the region. For example, each region can extend into a third dimension in the case where the substrate is porous while not having any substantial third dimension measurement (thickness) in the case where the substrate is non-porous. An array can contain more than ten, more than one hundred, more than one thousand more than ten thousand features, or even more than one hundred thousand features, in an area of less than 20 cm² or even less than 10 cm². For example, features can have widths (that is, diameter, for a round spot) in the range of from about 10 μm to about 1.0 cm. In other embodiments each feature can have a width in the range of about 1.0 μm to about 1.0 mm, such as from about 5.0 μm to about 500 μm, and including from about 10 μm to about 200 μm. Non-round features can have area ranges equivalent to that of circular features with the foregoing width (diameter) ranges. A given feature is made up of chemical moieties, e.g., nucleic acids, that bind to (e.g., hybridize to) the same target (e.g., target nucleic acid), such that a given feature corresponds to a particular target. At least some, or all, of the features are of different compositions (for example, when any repeats of each feature composition are excluded the remaining features can account for at least 5%, 10%, or 20% of the total number of features). Interfeature areas will typically (but not essentially) be present which do not carry any polynucleotide. Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature can incidentally detect non-targets of that feature). The target for which each feature is specific is, in representative embodiments, known. An array feature is generally homogenous in composition and concentration and the features can be separated by intervening spaces (although arrays without such separation can be fabricated).

The phrase “oligonucleotide bound to a surface of a solid support” or “probe bound to a solid support” or a “target bound to a solid support” refers to an oligonucleotide or mimetic thereof, e.g., PNA, LNA or UNA molecule that is immobilized on a surface of a solid substrate, where the substrate can have a variety of configurations, e.g., a sheet, bead, particle, slide, wafer, web, fiber, tube, capillary, microfluidic channel or reservoir, or other structure. In some embodiments, the collections of oligonucleotide elements employed herein are present on a surface of the same planar support, e.g., in the form of an array. It should be understood that the terms “probe” and “target” are relative terms and that a molecule considered as a probe in certain assays can function as a target in other assays.

“Addressable sets of probes” and analogous terms refer to the multiple known regions of different moieties of known characteristics (e.g., base sequence composition) supported by or intended to be supported by an array surface, such that each location is associated with a moiety of a known characteristic and such that properties of a target moiety can be determined based on the location on the array surface to which the target moiety binds under stringent conditions.

An “array layout” or “array characteristics”, refers to one or more physical, chemical or biological characteristics of the array, such as positioning of some or all the features within the array and on a substrate, one or more feature dimensions, or some indication of an identity or function (for example, chemical or biological) of a moiety at a given location, or how the array should be handled (for example, conditions under which the array is exposed to a sample, or array reading specifications or controls following sample exposure).

In some embodiments, an array is contacted with a nucleic acid sample under stringent assay conditions, i.e., conditions that are compatible with producing bound pairs of biopolymers of sufficient affinity to provide for the desired level of specificity in the assay while being less compatible to the formation of binding pairs between binding members of insufficient affinity. Stringent assay conditions are the summation or combination (totality) of both binding conditions and wash conditions for removing unbound molecules from the array.

As known in the art, “stringent hybridization conditions” and “stringent hybridization wash conditions” in the context of nucleic acid hybridization are sequence dependent, and are different under different experimental parameters. Stringent hybridization conditions include, but are not limited to, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization in 0.5 M NaHPO₄, 7% sodium dodecyl sulfate (SDS), 1 mM EDTA at 65° C., and washing in 0.1×SSC/0.1% SDS at 68° C. can be performed. Additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature. Other methods of agitation can be used, e.g., shaking, spinning, and the like.

Stringent hybridization conditions can also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and can also be employed, as appropriate. The term “highly stringent hybridization conditions” as used herein refers to conditions that are compatible to produce complexes between complementary binding members, i.e., between immobilized probes and complementary sample nucleic acids, but which do not result in any substantial complex formation between non-complementary nucleic acids (e.g., any complex formation which cannot be detected by normalizing against background signals to interfeature areas and/or control regions on the array).

Additional hybridization methods are described in Kallioniemi et al. (1992) Science 258:818-821 and WO 93/18186. Several guides to general techniques are available, e.g., Tijssen, Hybridization with Nucleic Acid Probes, Parts I and II (Elsevier, Amsterdam, 1993). For descriptions of techniques suitable for in situ hybridizations see, Gall et al. (1981) Meth. Enzymol. 21:470-480 and Angerer et al., In Genetic Engineering: Principles and Methods, Setlow and Hollaender, Eds. Vol 7, pgs 43-65 (Plenum Press, New York, 1985). See also U.S. Pat. Nos.: 6,335,167; 6,197,501; 5,830,645; and 5,665,549; the disclosures of which are herein incorporated by reference.

After hybridization, the array can be subjected to washing procedures. The hybridized array may be washed in solutions having the same or different stringency as the hybridization. In some embodiments, a hybridized array may be washed at successively higher stringency solutions. Wash conditions used to remove unbound nucleic acids can include, in some embodiments, a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex can be washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Conditions for washing can also be, e.g., 0.2×SSC/0.1% SDS at 42° C. Other non-limiting examples of conditions for washing are described hereinbelow.

In the case of an array, the “target” will be referenced as a moiety in a mobile phase (typically fluid), to be detected by probes (“target probes”) which are bound to the substrate at the various regions. However, either of the “target” or “target probes” may be the one which is to be detected by the other (thus, either one could be an unknown mixture of polynucleotides to be detected by binding with the other). “Addressable sets of probes” and analogous terms refer to the multiple regions of different moieties supported by or intended to be supported by the array surface.

The term “sample” as used herein relates to a material or mixture of materials, containing one or more components of interest. Samples include, but are not limited to, samples obtained from an organism or from the environment (e.g., a soil sample, water sample, etc.) and can be directly obtained from a source (e.g., such as a biopsy or from a tumor) or indirectly obtained e.g., after culturing and/or one or more processing steps. In some embodiments, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

“Substrate” or “substrate surface” refers to a porous or non-porous water insoluble support material. The substrate can have any one of a number of shapes, such as strip, plate, disk, rod, particle, including bead, and the like. The substrate surface can be hydrophobic or hydrophilic or capable of being rendered hydrophobic or hydrophilic and can include, for example, inorganic powders such as silica, magnesium sulfate, and alumina; natural polymeric materials, particularly cellulosic materials and materials derived from cellulose, such as fiber-containing papers, e.g., filter paper, chromatographic paper, and like materials; synthetic or modified naturally occurring polymers, such as nitrocellulose, cellulose acetate, poly(vinyl chloride), polyacrylamide, cross linked dextran, agarose, polyacrylate, polyethylene, polypropylene, poly(4-methylbutene), polystyrene, polymethacrylate, poly(ethylene terephthalate), nylon, poly(vinyl butyrate), and like materials; either used alone or in conjunction with other materials; glass available as Bioglass, ceramics, metals, and like materials. Natural or synthetic assemblies such as liposomes, phospholipid vesicles, and cells can also be employed.

In some embodiments, substrates used for arrays in accordance with the present disclosure are surface-derivatized glass or silica, or polymer membrane surfaces, as described in Z. Guo, et al., Nucleic Acids Res, 22, 5456-65 (1994); U. Maskos, E. M. Southern, Nucleic Acids Res, 20, 1679-84 (1992), and E. M. Southern, et al., Nucleic Acids Res, 22, 1368-73 (1994), each incorporated by reference herein. In modifying siliceous or metal oxide surfaces, one technique that has been used is derivatization with bifunctional silanes, i.e., silanes having a first functional group enabling covalent binding to the surface (often an Si-halogen or Si-alkoxy group, as in —SiCl₃ or —Si(OCH₃)₃, respectively) and a second functional group that can impart the desired chemical and/or physical modifications to the surface to covalently or non-covalently attach ligands and/or the polymers or monomers for the biological probe array. Silylated derivatizations and other surface derivatizations that are known in the art are within the scope of the disclosure. See for example U.S. Pat. No. 5,624,711 to Sundberg, U.S. Pat. No. 5,266,222 to Willis, and U.S. Pat. No. 5,137,765 to Farnsworth, each incorporated by reference herein. Other processes for preparing arrays are described in U.S. Pat. No. 6,649,348, to Bass et. al., assigned to Agilent Corp., which disclose DNA arrays created by in situ synthesis methods.

Immobilization of oligonucleotides on a substrate or surface can be accomplished by well-known techniques, commonly available in the literature. See for example A. C. Pease, et al., Proc. Nat. Acad. Sci, USA, 91:5022-5026 (1994); Z. Guo, et al., Nucleic Acids Res, 22, 5456-65 (1994); and M. Schena, et al., Science, 270, 467-70 (1995), each incorporated by reference herein.

“Siliceous substrate” refers to any material largely comprised of silicon dioxide. Silylated siliceous substrate is a siliceous substrate that has at least one surface derivatized with a silane compound using materials and methods known in the art to facilitate the bonding of nucleic acid probes.

“Bubble” refers to a small ball of gas in a fluid. The word “bubble” used alone encompasses both a gas bubble and a vapor bubble.

“Set” or “sub-set” of any item, such as a set of proteins or peptides, may contain only one of the item, or only two, or three, or any multiple number of the items.

A “peptide mixture” is typically a complex mixture of peptides obtained as a result of the cleavage of a sample comprising proteins.

A “sample of proteins” is typically any complex mixture of proteins and/or their modified and/or processed forms, which may be obtained from sources, including, without limitation: a cell sample (e.g., lysate, suspension, collection of adherent cells on a culture plate, a scraping, a fragment or slice of tissue, a tumor, biopsy sample, an archival cell or tissue sample, laser-capture dissected cells, and like sources), an organism (e.g., a microorganism such as a bacteria or yeast), a subcellular fraction (e.g., comprising organelles such as nuclei or mitochondria, large protein complexes such as ribosomes or golgi, and like sources), an egg, sperm, embryo, a biological fluid, viruses, and like sources.

“Peptide” refers to an entity comprising at least one peptide bond, and can comprise either D and/or L amino acids. A peptide can have, for example, about 2 to about 20 amino acids (e.g., about 2, 3, 4, 5, 6, 7, 8, 9, or 10 amino acids).

“Protein” refers to any protein, including, but not limited to peptides, enzymes, glycoproteins, hormones, receptors, antigens, antibodies, growth factors, and like natural or synthetic molecules, without limitation. Proteins include those comprised of greater than about 20 amino acids, greater than about 35 amino acid residues, or greater than about 50 amino acid residues.

“Peptide,” “polypeptide,” and “protein” are generally used interchangeably herein.

A “biological fluid” includes, but is not limited to, for example, blood, plasma, serum, sputum, urine, tears, saliva, sputum, cerebrospinal fluid, ravages, leukapheresis samples, milk, ductal fluid, perspiration, lymph, semen, umbilical cord fluid, and amniotic fluid, as well as fluid obtained by culturing cells, such as fermentation broth and cell culture medium.

A “sample of complex proteins” may contain, for example, greater than about 100, about 500, about 1,000, about 5,000, about 10,000, about 20,000, about 30,000, about 100,000 or more different proteins. Such samples may be derived from a natural biological source (e.g., cells, tissue, bodily fluid, soil or water sample, and the like) or may be artificially generated (e.g., by combining one or more samples of natural and/or synthetic or recombinant sources of proteins).

“Expression” refers to a level, form, or localization of product. For example, “expression of a protein” refers to one or more of the level, form (e.g., presence, absence or quantity of modifications, or cleavage or other processed products), or localization of the protein.

“Proteome” refer to the protein constituents expressed by a genome, typically represented at a given point in time. A “sub-proteome” is a portion or subset of the proteome, for example, the proteins involved in a selected metabolic pathway, or a set of proteins having a common enzymatic activity.

A “remote location,” refers to location other than the location at which the hybridization and/or array analysis occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating information” refers to transmitting the data representing that information as signals (e.g., electrical, optical, radio, magnetic, etc) over a suitable communication channel (e.g., a private or public network).

A component of a system which is “in communication with” or “communicates with” another component of a system receives input from that component and/or provides an output to that component to implement a system function. A component which is “in communication with” or which “communicates with” another component may be, but is not necessarily, physically connected to the other component. For example, the component may communicate information to the other component and/or receive information from the other component. “Input” or “output” may be in the form of electrical signals, light, data (e.g., spectral data), materials, or may be in the form of an action taken by the system or component of the system. The term “in communication with” also encompasses a physical connection that may be direct or indirect between one system and another or one component of a system and another.

“Forwarding” an item refers to any means of getting that item from one location to the next, whether by physically transporting that item or otherwise (where that is possible) and includes, at least in the case of data, physically transporting a medium carrying the data or communicating the data.

A “computer-based system” refers to the hardware means, software means, and data storage means used to analyze the information of the present disclosure. The minimum hardware of the computer-based systems of the present disclosure comprises a central processing unit (CPU), input means, output means, and data storage means. A skilled artisan can readily appreciate that any one of the currently available computer-based system are suitable for use in the present disclosure. The data storage means may comprise any manufacture comprising a recording of the present information as described above, or a memory access means that can access such a manufacture. In certain instances a computer-based system may include one or more wireless devices.

To “record” data, programming or other information on a computer readable medium refers to a process for storing information, using any such methods as known in the art. Any convenient data storage structure may be chosen, based on the means used to access the stored information. A variety of data processor programs and formats can be used for storage, for example, word processing text file, database format, and like formats.

A “processor” refers to any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

A “database” is a collection of information or facts organized according to a data model, which determines whether the data is ordered using linked files, hierarchically, according to relational tables, or according to some other model determined by the system operator.

An “information management system” refers to a program, or series of programs, which can search a database and determine relationships between data identified as a result of such a search.

An “interface on the display of a user device” or “user interface” or “graphical user interface” is a display (comprising text and/or graphical information) displayed by the screen or monitor of a user device connectable to the network which enables a user to interact with a system processor and/or system memory (e.g., including a data base and information management system).

“Providing access to at least a portion of a database” refers to making information in the database available to user(s) through a visual or auditory means of communication.

“Separation” refers to dividing, partially or completely, a substance, such as a nucleotide or protein mixture, into its component parts, such as like similar protein molecules or complementary hybridization of a nucleotide or nucleotide mixture, and optionally the removal of impurities. “Separation” can also refer to resolution of a signal peak, for example, from a near-by signal, from noise, or combinations thereof.

“Assessing” and “evaluating” are used interchangeably to refer to any form of measurement, and includes determining if an element is present or not.

“Determining,” “measuring,” “assessing,” “assaying” and like terms are used interchangeably and can include both quantitative and qualitative determinations. Assessing may be relative or absolute. “Assessing the presence of” includes determining the amount of something present, as well as determining whether it is present or absent.

It must be noted that as used herein and in the appended claims, the singular forms “a”, “and”, and “the” include plural referents unless the context clearly dictates otherwise. Thus, for example, reference to “a protein” can include a plurality of such proteins and reference to “protein” can include reference to one or more proteins and equivalents thereof known to those skilled in the art.

“About” modifying, for example, the quantity of an ingredient in a composition, concentrations, volumes, process temperatures, process times, recoveries or yields, flow rates, and like values, and ranges thereof, employed in describing the embodiments of the disclosure, refers to variation in the numerical quantity that can occur, for example, through typical measuring and handling procedures; through inadvertent error in these procedures; through differences in the ingredients used to carry out the methods; and like proximate considerations. The term “about” also encompasses amounts that differ due to aging of a formulation with a particular initial concentration or mixture, and amounts that differ due to mixing or processing a formulation with a particular initial concentration or mixture. Whether modified by the term “about” the claims appended hereto include equivalents to these quantities.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

Generally, nucleic acid hybridizations can comprise the following major steps: (1) immobilization of probe nucleic acid sequences; (2) prehybridization treatment to increase accessibility of target DNA, and to reduce nonspecific binding; (3) hybridization of the mixture of nucleic acid sequences to the nucleic acid on the solid surface; (4) posthybridization washes to remove nucleic acid fragments not bound in the hybridization and (5) detection of the hybridized nucleic acid fragments. The reagent used in each of these steps and their conditions for use vary depending on the particular application. Polynucleotide microarrays can be washed after hybridization to remove labeled target molecules that are not specifically hybridized to their corresponding probes.

The present disclosure is based in part on the surprising discovery by Applicant that the addition of a superwetting agent to a solution used in a washing procedure after a hybridization step led to various improvements and benefits as illustrated herein. The present disclosure provides, in some embodiments, an improved washing composition which can reduce the incidence of non-uniform features on microarrays and reduce the incidence of non-uniform backgrounds on microarrays.

Buffers used to wash microarrays are typically salt solutions, the concentrations of which can define the stringency of the system and the quality of the data. If the stringency is too high, specifically hybridized targets will be washed from the microarray leading to the loss of signal and therefore, lower signal-to-noise rations. Conversely, low-stringency conditions will lead to non-specific probe binding and masking of specific signal. Without wishing to be bound by any particular theory, it is believed that he rate at which microarrays are dried can also impact the quality of the resultant data. When a large force (high surface tension, high gas pressure, etc.) is used to sheet the wash buffer from the microarray, the rapid drying of droplets remaining on the polynucleotide features increases the risk that this drying will be non-uniform. Non-uniform drying can result in non-uniform distribution of labeled target within the feature that can compromise the quality of data extracted from that feature. Conversely, if wash buffers dry too slowly, large droplets of buffer may dry to deposit salts on the surface, resulting in areas of elevated backgrounds or non-uniform backgrounds. It is believed that addition of superwetting agents to microarray wash buffers can improve the uniformity of array drying by lowering the surface tension of the liquid to allow easier spreading.

Particularly preferred superwetting agents of the disclosure are those compounds or compositions that are water soluble or dispersible that can be used with water-based systems to impart very high wetting and very low surface tension to the contacted surface. In some embodiments, concentrations of superwetting agent in the contacting formulations can range, for example, from about 0.01% to 50% by weight or volume, from about 0.05% to 20% by weight or volume, and from about 0.1% to 10% by weight or volume, based on the total weight (w/w) or volume (w/v) of the wash buffer composition. In some embodiments, concentrations of superwetting agent can vary from 0.001% to 2%; from 0.001% to 1%; or from 0.005% to 0.02% by weight or volume.

Superwetting agents useful in the present methods and compositions include, for example, silicone based surfactants such as silicone polyoxyalkylene copolymers. Such materials are commercially available, for example, as TEGO® Wet 260, TEGO® Wet 280, TEGO® Wet KL 245. Superwetting polyether siloxane copolymers, or alternatively referred to as silicone polyoxyalkylene copolymers, such as TEGO® Wet 260, are commercially available from Tego Chemie Service, GmbH, formerly Goldschmidt Chemicals. Other examples of silicone surfactant as superwetting can be found in Silicone Surfactants, R. M. Hill, ed., Marcel Dekker, 1999. Other suitable superwetting agents include, for example, fluorocarbon surfactants, such as Zonyl® and Novec®, and the hydrocarbon-based surfactants, such as TEGO® Wet 510, Dynol®, Surfynol®, and EnviroGem® surfactants.

Superwetting agents such as TEGO® Wet 260 are miscible with water in all concentrations at room temperature. However, at the elevated temperatures that are sometimes required for DNA array hybridization, for example 65° C., phase separation of the TEGO® Wet 260 surfactant from the aqueous buffer may be observed depending upon, for example, the amount of the superwetting agent, the presence or absence of other components in the buffer composition, and like considerations. The presence of an additional surfactant such as lithium dodecyl sulfate (LiDS) can be used to prevent this phase separation. The amount of LiDS required to prevent this phase separation is a function of the amount of TEGO® Wet 260 added to the buffer composition. In some embodiments, suitable formulations can be formulated to contain sufficient LiDS, or an equivalent material, to prevent phase separation at, for example, about 65° C.

In some embodiments, the superwetting agent can comprise an organosilicone wetting agent, an organofluorine wetting agent, a hydrocarbyl wetting agent, and like superwetting materials, or mixtures thereof. In some embodiments, superwetting agents employed in the present methods and compositions include, for example, certain organosilicone-based surfactants, for example, having the general formula:

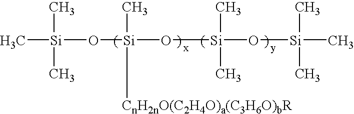

wherein x is from about 1 to about 10; y is from about 0 to about 10; n is from about 3 to about 4; a is from about 0 to about 15; b is from about 0 to about 14; such that at least one of a and b is not zero and a+b is from about 1 to about 30; and R can be, for example, hydrogen, an alkyl group having from about 1 to about 4 carbon atoms, an acetyl group, and mixtures thereof. Other like structural variants are believed to be suitable, such as where the alkoxylation can be, additionally or alternatively, in the organosilicone copolymer backbone.

Examples of specific superwetting agents are disclosed in U.S. Pat. No. 6,503,413, to Uchiyama, et al., issued Jan. 7, 2003, such as a polyalkyleneoxide polysiloxane having the formula:

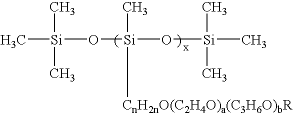

wherein x is from about 1 to about 8; n is from about 3 to about 4; a is from about 1 to about 15; b is from about 0 to about 14; a+b is from about 5 to about 15; and R is selected from the group consisting of hydrogen, an alkyl group having from about 1 to about 4 carbon atoms, and an acetyl group. The polyalkylene polysiloxane can have a molecular weight of from about several hundred to many thousands, for example, less than about 1,000 to 10,000.

Still other superwetting agents suitable for use in the present method and composition include certain organosilicone-based and fluorocarbon-based surfactants, as disclosed for example in U.S. Pat. No. 5,985,793, to Sandbrink, et al., issued Nov. 16, 1999, such as a polyalkyleneoxide polysiloxane having the formula:

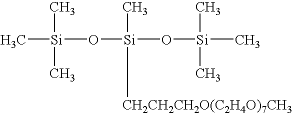

and like polyether siloxane copolymers.

U.S. Pat. No. 4,380,451, to Steinberger, issued Apr. 19, 1983, discloses continuous dyeing and simultaneous finishing of textile materials using defoaming agent of polyoxyalkylene polysiloxane copolymer and hydrophobic silica, including a method for preparing the polyoxyalkylene polysiloxane copolymer material, see Example I. U.S. Pat. No. 4,728,457, to Fieler, et al., issued Mar. 1, 1988, discloses non-volatile silicone fluids that can be, for example, a polyalkyl siloxane, a polyaryl siloxane, a polyalkylaryl siloxane, or a polyether siloxane copolymer. Other disclosures of suitable silicone fluids include U.S. Pat. No. 2,826,551, to Geen, U.S. Pat. No. 3,964,500, to Drakoff, U.S. Pat. No. 4,364,837, to Pader, and British Pat. No. 849,433, to Woolston. All of these patents are incorporated herein by reference. Also incorporated herein by reference is Silicon Compounds distributed by Petrarch Systems, Inc., 1984. Another source of siloxanes, silicones, and related compounds, useful in the present disclosure is, for example, Gelest, Inc., reference the 1995 catalog and www.gelest.com. Still other silicone material that can be useful in the present compositions include silicone gums described by Petrarch and others, including U.S. Pat. No. 4,152,416, Spitzer et al., and Noll, Walter, Chemistry and Technology of Silicones, N.Y.: Academic Press 1968. U.S. Pat. No. 5,156,834, to Beckmeyer, et al., discloses antiperspirant compositions including a non-volatile emollient such as a polyalkyl siloxane, a polyalkylaryl siloxane or a polyether siloxane copolymer. These siloxanes are available, for example, from the General Electric Company as the Vicasil series and from Dow Corning as the Dow Corning 200 series. EP-A 633 018 discloses the use of polyoxyalkylenepolydimethylsiloxanes and like copolymers can be prepared, for example, by hydrosilylation in the presence of a platinum-containing catalyst. See also J. B. Plumb and J. H. Atherton, Block Copolymers, publisher; D.C. Allport and W. H. Janes, Applied Science Publishers Ltd., London, 1973, page 305-325. Copolymers are known in which polyoxyalkylene groups, as well as long-chain hydrocarbon groups are linked to a linear polysiloxane. The synthesis of such compounds is described in U.S. Pat. Nos. 3,234,252, 4,047,958, 4,427,958, 3,427,271 and 2,846,458. The synthesis can be accomplished by adding an olefin with, for example, 6 to 18 carbon atoms, and a polyoxyalkylene ether of an olefinically unsaturated alcohol, e.g., the polyoxyalkylene ether of allyl alcohol, to a polydiorganosiloxane having SiH groups, the addition being carried out in the presence of a catalyst containing platinum. Ethoxylated organosilicone wetting agents are also disclosed in U.S. Pat. Nos. 5,985,793, 4,160,776, 4,226,794, and 4,337,168, the disclosures of which are incorporated herein by reference.

Still other examples of suitable polyalkyleneoxide polysiloxane surfactants are commercially available under the trade names Silwet® L-77, Silwet® L-7280, and Silwet® L-7608 available from Witco Corporation; and DC Q2-5211 and Sylgard® 309 available from Dow Corning Corporation.

Fluorocarbon-based superwetting agent can be, for example, an organofluorine compound of the formulas:

a) {(CₙF₂ₙ₊₁—C_{n′}H_{2n′})ₚ—SOₘ—R}ₓ

wherein

m is from 1 to 3; n is from about 3 to about 20; n′ is from about 0 to about 20; p is from 1 to 2; x is from about 1 to about 100; R is hydrogen, (C₁₋₁₀) saturated alkyl, or (C₂₋₁₀₎ unsaturated alkyl; or salts thereof;

b) (CₙF₂ₙ₊₁—C_{n′}H₂ₙ)ₚR¹

wherein

R¹ is monovalent or divalent radical selected from the group

—H,

halo, such as —CL, —Br, —I,

—OH,

(C₁₋₁₀) alkyl,

(C₂₋₁₀) unsaturated alkyl,

—CH₂2-O—C(═O)—(C₁₋₂₀) alkyl,

—CH₂—O—C(═O)—(C₂₋₂₀) unsaturated alkyl,

—O—P(═O)(OH)₂,

—O—P(═O)(—OH)—,

—O—P(═O)(—OH)(—O—CH₂—CH₂—)ₒ—OH,

—CO₂H,

—⁽⁺⁾N(R²)₂—O⁽⁻⁾,

—⁽⁺⁾N(R²)—CH₂—CO₂H,

—O—C(═O)—CH₂—CH(SO₃H)—C(═O)—O—,

—O(CH₂—CH₂—O)_{y}—H,

—S—CH₂—CH₂—C(═O)—OH, or

(C₁₋₁₀)alkyl-C(═O)—O—CH—CH₂—⁽⁺⁾N(R²)₂—(CH₂)₁₋₄—CO₂H,

each R² is independently hydrogen, or (C₁₋₁₀) alkyl,

m is from 1 to 2;

n is from about 1 to about 20;

n′ is from about 1 to about 20;

o is from about 1 to about 10;

p is from 1 to 2; and

y is from 0 to about 20;

or salts thereof;

c)

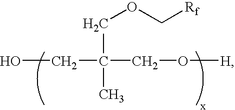

wherein

R_{f} is a monovalent radical of the formula —CₙF₂₊₁;

n is from about 1 to about 20; and

x is from 1 to 100;

or salts thereof,

and like or equivalent compounds including polymers thereof where compounds a), b), or c) can be polymerized, and mixtures thereof.

Compounds of the above formula a) are known, for example, as Novec™ fluorosurfactants and are available from 3M Company. Compounds of the formula b) are known, for example, as Marsurf FS-fluorosurfactants and are available from Mason Chemical Company, and as Zonyl® and Forafac® fluorinated surfactants or intermediates, and are available from DuPont Company. Compounds of the formula c) are known as PolyFox products and are available from OMNOVA Solutions, Inc. (www.omnova.com).

Preferred compounds of the formula CₙF₂ₙ₊₁—SOₘR can be, for example, non-ionic and anionic sulfonyl compounds and polymers thereof, and which products are commercially available as Novec™ fluorosurfactants (replacing 3M Fluorad™ fluorosurfactants) from 3M Company, such as the perflurobutanesulfonates. In the formula CₙF₂ₙ₊₁—SOₘR the carbon chain in CₙF₂ₙ₊₁ and the (C₁₋₁₀) alkyl or (C₂₋₁₀) unsaturated alkyl selections for R, as with other alkyl substituents of the disclosure, can be linear, branched, cyclic, or mixtures thereof. Specific Novec™ superwetting materials include non-ionic polymeric fluorosurfactant products FC-4432 and FC-4430.

Still other superwetting agents of the disclosure can be, for example, hydrocarbon-based or hydrocarbyl wetting agent, such as an alkoxylated hydrocarbyl compound of the formula:

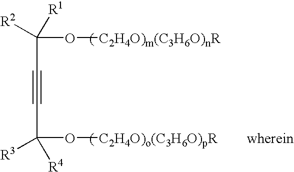

R is hydrogen or an alkyl radical having from 1 to 4 carbon atoms,

R¹ to R⁴ are each independently hydrogen or an alkyl radical having from 3 to 10 carbon atoms; and m, n, o and p are from 0 to 30 and a sum of 3 to about 60, inclusive. The alkoxylated copolymer units comprised of m and o units which represent ethylene oxide adducts (EO) and n and p units which represent propylene oxide adducts (PO), when either or both m+n or o+p are present, can be, for example, random EO—PO mixtures, block EO—PO, gradient EO—PO mixtures, alternating EO—PO mixtures, segmented EO—PO mixtures, and like combinations, or mixtures thereof. The alkoxylated copolymer units represented by m and n, and o and p, can be in any order, such as —O-(EO)ₘ—(PO)ₙ—R or —O(PO)ₙ+(EO)ₘ—R. U.S. Pat. No. 6,313,182, assigned to Air Products, discloses an acetylenic diol ethylene oxide/propylene oxide adduct for example, an acetylenic glycol compound or alternatively referred to as an ethoxylated acetylenic diol compound available as Dynol™ surfactants from Air Products Corporation, such as Dynol™ 604 and ethoxylated and ethoxylated/propoxylated products such as Surfynol™, and EnviroGem™ AE Surfactants. The following U.S. patents also describe various acetylenic alcohols and their ethoxylates as surface active agents: U.S. Pat. No. 3,268,593 (ethylene oxide adducts of tertiary acetylenic alcohols); U.S. Pat. No. 4,117,249 (3 to 30 mole ethylene oxide (EO) adducts of acetylenic glycols); and U.S. Pat. No. 5,650,543. Specific acetylenic diol-ethylene oxide adducts include the ethylene oxide adducts of, for example, 3-methyl-1-nonyn-3-ol, 7,10-dimethyl-8-hexadecyne-7,10-diol; 2,4,7,9-tetramethyl-5-decyne-4,7-diol and 4,7-dimethyl-5-decyne-4,7-diol.

Yet still other superwetting agents of the disclosure can be any other similar or any other proprietary material which can provide the desired wetting characteristics and hybridization results, for example, EnviroGem™ defoaming wetting agents, such as AD01, commercially available from Air Products Corp., which have a dual hydrophobe and dual hydrophile structure (i.e., a dimeric or gemini surfactant).

The buffer capacity and the pH of the aqueous mixture containing the superwetting agent can be at, for example, from about 5 to about 9, from about 5 to about 8, from about 5 to about 7, from about 5 to about 6, and like pH values, such as from about 5.5 to about 6.7, in some embodiments. The contacting can be accomplished at various temperatures known to those skilled in the art, for example, from about 30° C. to about 70° C., from about 40° C. to about 70° C., from about 50° C. to about 70° C., from about 50° C. to about 65° C., from about 55° C. to about 65° C., and like temperatures.

In some embodiments, methods and compositions of the present disclosure can include in the aqueous mixture, in addition to one or more superwetting agents, additional performance enhancing components, for example, one or more of: an organosulfonic acid; an organosulfate surfactant; an organopolyalkoxylate; a source of a monovalent cation; a chelator; or mixtures thereof. In some embodiments, compositions of the present disclosure can include a buffer, or a buffer concentrate, comprising: a superwetting agent and a buffer component. The buffer or buffer concentrate can comprise any suitable buffer such as an organic compound, an inorganic compound, or mixtures thereof, and as illustrated herein.

The buffer concentrate or buffer concentrate can, for example, further comprise one or more of:

a source of a monovalent cation;

a water soluble organosulfonic acid or salt thereof;

a chelator or salt thereof;

an organosulfate surfactant or salt thereof;

an organopolyalkoxylate;

an aqueous carrier;

or mixtures thereof.

The source of a monovalent cation can be, for example, an alkali metal halide salt, such as lithium, sodium, potassium halide salts, e.g., LiCl, and like salts, or mixtures thereof. The organosulfonic acid can be, for example, a morpholino-substituted alkyl sulfonic acid, such as MES, MOPS, and like materials. The chelator can be, for example, a divalent cation chelating agent, such as EDTA and like chelators, or mixtures thereof. The organosulfate surfactant can be, for example, a (C₈-C₁₆) alkyl sulfate, such as a sodium or lithium dodecylsulfate (LiDS)(laurylsulfate, n-C₁₂H₂₅—OSO₃′), and like surfactants, or mixtures thereof. The organopolyalkoxylate can be, for example, an alkoxylated alkylphenol, such as Triton X-100, 4-octylphenol polyethoxylate, molecular formula: C₁₄H₂₂O(C₂H₄O)ₙH where the average number of ethylene oxide units per molecule is around 9 or 10 (CAS No: 9002-93-1), and like materials, or mixtures thereof.

In some embodiments of a wash buffer or wash buffer concentrate, the superwetting agent can be, for example, present in from about 0.001 to about 50 weight percent. An water soluble organosulfonic acid can be present in from about 0.001 to about 10 weight percent. An organosulfate surfactant can be, for example, present in from about 0.001 to about 50 weight percent. An organopolyalkoxylate can be, for example, present in from about 0.001 to about 50 weight percent. A chelator can be, for example, present in from about 0.001 to about 10 weight percent of the total concentrate. A monovalent cation can be, for example, present in from about 0.01 to about 50 weight percent. An aqueous carrier, such as water, can be, for example, absent or minimized in the wash buffer or concentration, or provided to balance the buffer or composition to achieve convenient storage or use concentrations, for example, 1× use concentrations, and 2×, 3×, 4×, 5×, and like nx. concentrates where n is a multiplier integer or a fraction thereof. In embodiments, the aqueous carrier can include other ingredients, for example, dissolved urea, formamide, and like compounds or co-solvents. In embodiments, the carrier can alternatively comprise non-aqueous media, for example, formamide, DMSO, and like liquids. The compositions of the present disclosure, in some embodiments, can comprise an aqueous carrier that comprises water. The water used can be distilled, deionized, or tap water. The amount of water in the present compositions can vary dependent upon the specific uses of the composition. The component parts of the formulation add up to 100 weight percent or alternatively parts by weight. A wash buffer can have, for example, a pH from about 4 to 10, and useful compositions can have any suitable intermediate pH and as illustrated herein.

The present disclosure provides methods and compositions for treating nucleic acid microarrays after hybridization with nucleic acid materials, and can be used, for example, in high throughput analytical, therapeutic, and diagnostic applications. In some embodiments, the methods use post-hybridization treatment conditions, which are advantageously compatible with siliceous substrates and surface-derivatized siliceous substrates, for performing assays, for example, at high washing and/or soaking temperatures for long periods of time. In embodiments, the substrates are hydrophobic, such as polymer coated surfaces, see for example, U.S. Pat. Nos. 6,444,268, and 6,258,454, to Lefkowitz et. al., assigned to Agilent Corp., which patents disclose silane chemistry and surface modification for creating substrates for DNA arrays having moderately hydrophobic surfaces. In some embodiments, the present disclosure provides washing and/or soaking compositions and conditions that work particularly well on, for example, silane-derivatized siliceous substrates (“silylated-siliceous” substrates). The washing and soaking conditions of the present disclosure can include solution pH, buffer type, salt composition, surfactant composition, temperature, and time. The present disclosure promotes sensitive and selective detection of nucleic acid targets, while preserving the integrity and stability of the derivatized siliceous surface.

In some aspects of the disclosure, there are provided methods of treatment of hybrids formed on microarrays. In some embodiments, the methods comprise the steps of maintaining a wash solution at a pH between pH 5 and 7, and can include maintaining the temperature within the range from about 20° C. to about 70° C., from about 23° C. to about 60° C. or in the range of about 23° C. to about 55° C., for example. In some embodiments of the method of washing, the washing solution is maintained at a pH between pH 5.5 and 6.7 with a buffer having buffering capacity between pH 5.5 and 6.7, and having a monovalent cation present. In some embodiments, the pH is maintained between pH 6.0 and 6.6; the buffer is selected from one or more of SSC, SSPE, Tris-Cl, MES or MOPS; and the monovalent cation is provided by a salt preferably selected from one or more of LiCl, NaCl, or KCl. The wash temperature can range from about 23° C. to 55° C. In some embodiments, the wash buffer composition can further comprise one or more of a chelating agent and a surfactant.

In some embodiments, a wash buffer or buffer concentrate comprises SSC, SSPE or Tris-Cl. For example, SSC buffer (sodium chloride/sodium citrate 20×) is available commercially (Serva), and comprises: 3M NaCl (175 g/l); 0.3 M Na₃ citrate-2H₂O (88 g/l); with pH adjusted with 1 M HCl. As another example, 20× SSPE Buffer (3.0 M Sodium Chloride, 0.2 M Sodium Hydrogen Phosphate, 0.02 M EDTA, pH 7.4) is available commercially (Ambion). In some embodiments, a wash buffer comprises: 0.06× SSPE, 0.005% (w/v) sarcosine, and 0.01% (v/v) TEGO® Wet 260. In some embodiments, a wash buffer comprises: 0.1× SSPE, 0.005% N-lauroylsarcosine, and 0.01% TEGO® Wet 260.

In some embodiments, there are provided herein wash buffers comprising a siloxane superwetting agent. The siloxane superwetting agent can be a polyether siloxane. The polyether siloxane can be a polyether siloxane copolymer. The wash buffer can comprise about 15 mM to about 6 M of a salt, a non-limiting example of which is NaCl. The wash buffer can comprise about 1 mM to about 0.6 M of a buffer salt. An exemplary buffer salt comprises sodium citrate or sodium phosphate. The wash buffer can comprise about 0.1 mM to about 0.04 M of a metal chelator, a non-limiting example of which comprises EDTA. The wash buffer can comprise an amino acid, a non-limiting example of which comprises sarcosine. In some embodiments, the siloxane surfactant can be present in the wash buffer at 0.0001% to about 2.0%; about 0.001% to about 0.1%; or about 0.005% to about 0.02%. In some embodiments, the polymer siloxane copolymer superwetting agent can be TEGO® Wet 260, TEGO® Wet 280, or TEGO® Wet KL 245. In some embodiments, the washing buffer comprises 0.001% to about 0.1% of TEGO® Wet 260, TEGO® Wet 280, or TEGO® Wet KL 245.

In some embodiments, a wash buffer comprises about 0.006× to about 0.6× SSPE, about 0.0005% to about 0.05% sarcosine, and about 0.001% to about 0.1% of TEGO® Wet 260, TEGO® Wet 280, or TEGO® Wet KL 245. In some embodiments, a wash buffer comprises about 0.03× to about 0.12× SSPE, about 0.0025% to about 0.01% sarcosine, and about 0.005% to about 0.02% of TEGO® Wet 260, TEGO® Wet 280, or TEGO® Wet KL 245. In some embodiments, a wash buffer comprises about 0.06× SSPE, about 0.005% sarcosine, and about 0.01% of TEGO® Wet 260, TEGO® Wet 280, or TEGO® Wet KL 245.

A washing step, in some embodiments, can take anywhere from less than about 30 seconds to 10 minutes, from 1 minute to 5 minutes, or from 1 hour to more than 10 hours, for example. Multiple washes can be performed, at varied concentrations of buffer components, and at varied temperatures. There can be 1, 2, 3, 4, 5, or more wash steps, for example. In some embodiments, a post-hybridization processing procedure can comprise one or more washing steps. In some embodiments, a plurality of wash steps are employed, and a superwetting agent as described herein is used only in the last washing step prior to a drying step.

A washing step can be followed by a soaking step. A soaking step can utilize the same buffer as a preceding washing step, and can be carried out at the same temperature as a preceding washing step. A soaking step, in some embodiments, can take anywhere from less than about 30 seconds to 10 minutes, from 1 minute to 5 minutes, or from 1 hour to more than 10 hours, for example.

After the washing and optional soaking steps, the array can be dried using conventional methods (see, e.g., U.S. Pat. Nos. 6,858,186; 7,018,842 and U.S. Pat. Publication No. 20060234267). The drying may be accomplished using any suitable drying method and conditions which will not decompose the probes and their bound targets, such as any suitable one or more of: air drying at room temperature or raised temperature, reduced pressure; centrifuging; or exposure to a dry unreactive gas stream. Unreactive gases include nitrogen, noble gases, and the like, and mixtures thereof. Noble gases include, for example, helium, argon, krypton, xenon, neon, and the like. The gas can have a water content that is less than about 1 part per million (ppm) by volume, usually, less than about 0.1 ppm by volume. The gas can be present at any suitable pressure, such as, for example, in the range of 1-5 bar, or in the range of 2-4 bar:

Oligonucleotide hybridization can be performed utilizing essentially any conventional method. In some embodiments, a method of hybridizing a microarray of an oligonucleotide bound to a silylated-siliceous substrate with another oligonucleotide material can be used. In some embodiments, such methods of hybridizing comprise the steps of combining the oligonucleotide material with a hybridization buffer composition having a pH between pH 5.5 and 6.7 comprising a buffer and a monovalent cation; and incubating the material in the buffered composition with the oligonucleotide microarray at a hybridization temperature ranging from about 55° C. to about 70° C. to hybridize the oligonucleotide material. The incubation time period can range, for example, from less than about 2 hours to more than 48 hours. Preferably, the buffer can be selected from one or more of and organosulfonic acid such as MES or MOPS, which has a buffering capacity within the pH range of pH 5.5 and 6.7. The monovalent cation can be provided by a salt, such as an alkali halide, preferably selected from one or more of LiCl, NaCl, KCl, and mixtures thereof. The hybridization buffer composition can further comprise one or more of a superwetting agent, a chelating agent, and a surfactant (see, e.g., U.S. patent application Ser. No. 11/082,476).

A hybridization product can be the obtained in any of various analyses such as gene expression analysis (see, e.g., U.S. Pat. No. 6,927,032), comparative genomic hybridization experiments (see, e.g., Published U.S. Application No. 20060094022 and U.S. Pat. Nos. 7,011,949; 6,335,167; 6,197,501; 5,830,645; and 5,665,549), location analysis (see, e.g., U.S. Pat. No. 6,410,243), SNP detection or methylation analysis (see, e.g., WO2005123942), for example.

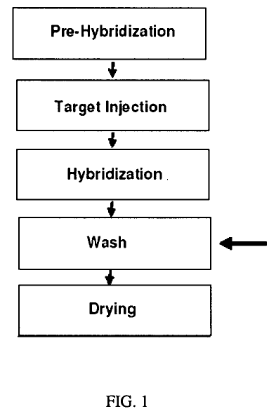

Some embodiments of methods as described herein are shown in FIG. 1 which shows a flow-chart diagram illustrating general steps in microarray hybridization procedures. In some embodiments, a superwetting agent can be included in a washing step as shown (arrow).

The methods and compositions of the present disclosure can overcome problems in the art as described herein.

In some aspects of the disclosure, kits are provided that comprise a microarray of an oligonucleotide on a derivatized surface of a siliceous substrate and instructions for performing a post-hybridization procedure (such as a washing step and/or soaking step) as described herein. The instructions can comprise the methods, the compositions, or both, of the present disclosure. In some embodiment, kits further comprise a washing buffer comprising a superwetting agent. The composition can comprise a buffer having a useful buffering capacity in that pH range.

The composition and preparative methods of the present disclosure can be used to prepare custom or made-to-order array articles, such as a specific unhybridized array or a specific hybridized array.

The buffer compositions of the present disclosure can also be used in an article of manufacture comprising the composition contained in a suitable container, dispenser, or combination thereof. Preferably the articles of manufacture are in association with instructions for how to use the composition in post-hybridization processing, including, for example, the manner, the amount, or both of the composition to use, and preferred ways to use. It is desirable that the instructions be as simple and clear as possible, so that using pictures or symbols may be desirable. Thus, a set of instructions can comprise an instruction to prepare a buffer concentrate or a working buffer, by following one or more of the described methods. A set of instructions can also comprise an instruction to use a buffer concentrate or a working buffer to treat a hybridized array. “In association with” refers to the set of instructions that can be either directly printed on the container or a container label, or presented in a separate manner including, but not limited to, a brochure, print advertisement, electronic advertisement, verbal communication, and like presentations, to communicate the set of instructions to a consumer or user of the article of manufacture. The set of instructions can comprise the instruction to apply an effective amount of the composition, for example, by contacting the working buffer with the hybridized array, to provide the indicated benefit.

The compositions can be packaged in a bottle, especially a bottle that comprises a measuring closure. The measuring closure provides a convenient way to dispense the appropriate amount of the composition, especially when dispensing concentrated compositions into a more dilute solution or mixture. The bottle can comprise a drain-back spout, which permits the composition to be dispensed more easily and with less waste or spillage. Non-limiting examples of suitable bottles are described in detail in U.S. Pat. No. 4,666,065, to Ohren; U.S. Pat. No. 4,696,416, to Muckenfuhs et al.; and U.S. Pat. No. 4,981,239, to Cappel et al.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the claims appended hereto.

Materials and Methods

Buffering Agents

The buffer compositions comprising a superwetting agent of the disclosure preferably include a buffering agent. The pH of the present compositions can be controlled within the ranges of from about 4 to about 10, from about 5 to about 9.5, or from about 6 to about 9. The buffering agent can be any organic or inorganic, acid or base, and alkali metal salts thereof, having at least one pKₐ value and/or pK_{b} value of from about 4 to about 10, from about 5 to about 9.5, or from about 6 to about 9. The buffering agent can be an alkali metal salt of an organic acid and/or inorganic acid having at least one pKₐ value of from about 6 to about 9. It is recognized that buffering agents may have more than one pKₐ value and/or pK_{b} value. A buffering agent can has at least one of its pKₐ values and/or pK_{b} values within the indicated ranges.

Suitable buffering agents can be, for example, acridine, phenylalanine, allothreonine, n-amylamine, aniline, n-allylaniline, 4-bromoaniline, 4-bromo-N,N-dimethylaniline, m-chloroaniline, p-chloroaniline, 3-chloro-N,N-dimethylaniline, 3,5-dibromoaniline, N,N-diethylaniline, N,N-dimethylaniline, N-ethylaniline, 4-fluoroaniline, N-methylaniline, 4-methylthioaniline, 3-sulfonic acid aniline, 4-sulfonic acid aniline, p-anisidine, arginine, asparagine, glycyl asparagine, DL-aspartic acid, aziridine, 2-aminoethylbenzene, benzidine, benzimidazole, 2-ethylbenzimidazole, 2-methylbenzimidazole, 2-phenylbenzimidazole, 2-aminobenzoic acid, 4-aminobenzoic acid, benzylamine, 2-aminobiphenyl, brucine, 1,4-diaminobutane, t-butylamine 4-aminobutyric acid, glycyl-2-amino-n-butyric acid, cacodylic acid, beta-chlortriethylammonium-n-butyric acid, codeine, cyclohexylamine, cystine, n-decylamine, diethylamine, n-dodecaneamine, 1-ephedrine, 1-amino-3-methoxyethane, 1,2-bismethylaminoethane, 2-aminoethanol, ethylenediamine, ethylenediaminetetraacetic acid, 1-glutamic acid, alpha-monoethylglutamic acid, 1-glutamine, 1-glutathione, glycine, n-acetylglycine, dimethylglycine, glycylglycylglycine, leucylglycine, methylglycine, phenylglycine, N-n-propylglycine, tetraglycylglycine, glycylserine, dexadecaneamine, 1-aminoheptane, 2-aminoheptane, 2-aminohexanoic acid, DL-histidine, beta-alanylhistidine, imidazol, 1-aminoindane, 2-aminoisobutyric acid, isoquinoline, 1-aminoisoquinoline, 7-hydroxyisoquinoline, 1-leucine, glycylleucine, methionine, methylamine, morphine, morpholine, 1-amino-6-hydroxynaphthalene, dimethylaminonaphthalene, alpha-naphthylamine, beta-naphthylamine, n-methyl-alpha-naphthylamine, cis-neobornylamine, nicotine, n-nonylamine, octadecaneamine, octylamine, ornithine, papaverine, 3-aminopentane, valeric acid, permidine, phenanthridine, 1,10-phenanthroline, 2-ethoxyaniline, 3-ethoxyaniline, 4-ethoxyaniline, alpha-picoline, beta-picoline, gamma-picoline, pilocarpine, piperazine, trans-2,5-dimethylpiperazine, 1-n-butylpiperidine, 1,2-dimethylpiperidine, 1-ethylpiperidine, 1-methylpiperidine, proline, hydroxyproline, 1-amino-2,2dimethylpropane, 1,2-diaminopropane, 1,3-diaminopropane, 1,2,3-triaminopropane, 3-aminopropanoic acid, pteridine, 2-amino4,6-dihydroxypteridine, 2-amino4-hydroxypteridine, 6-chloropteridine, 6-hydroxy4-methylpteridine, purine, 6-aminopurine, 2-dimethylaminopurine, 8-hydroxypurine, 2-methylpyrazine, 2-amino-4,6-dimethylpyrimidine, pyridine, 2-aldoximepyridine, 2-aminopyridine, 4-aminopyridine, 2-benzylpyridine, 2,5-diaminopyridine, 2,3-dimethylpyridine, 2,4-dimethylpyridine, 3,5-dimethylpyridine, 2-ethylpyridine, methyoxypyridine, 4-methylaminopyridine, 2,4,6-trimethylpyridine, 1,2-dimethylpyrrolidine, n-methylpyrrolidine, 5-hydroxyquinazoline, quinine, 3-quinolinol, 8-quinolinol, 8-hydroxy-5-sulfoquinoline, 6-methoxyquinoline, 2-methylquinoline, 4-methylquinoline, 5-methylquinoline, serine, strychnine, taurine, myristilamine, 2-aminothiazole, threonine, o-toluidine, m-toluidine, p-toluidine, 2,4,6-triamino-1,2,3-triazine, tridecaneamine, trimethylamine, tryptophan, tyrosine, tyrosineamide, valine, salts thereof, and mixtures thereof.

Other suitable buffering agents can be, for example, acetic acid, acetoacetic acid, acrylic acid, adipamic acid, adipic acid, d-alinine, allantoin acid, alloxanic acid, alpha-aminoacetic acid, o-aminobenzoic acid, p-aminobenzoic acid, m-aminobenzosulfonic acid, p-aminobenzosulfonic acid, anisic acid, o-beta-anisylpropionic acid, m-beta-propionic acid, p-beta-propionic acid, ascorbic acid, DL-aspartic acid, barbituric acid, benzoic acid, m-bromobenzoic acid, n-butyric acid, iso-butyric acid, cacodylic acid, n-caproic acid, iso-caproic acid, m-chlorobenzoic acid, p-chlorobenzoic acid, beta-chlorobutyric acid, gamma-chlorobutyric acid, o-chlorocinnamic acid, m-chlorocinnamic acid, p-chlorocinnamic acid, o-chlorophenylacetic acid, m-chlorophenylacetic acid, p-chlorophenylacetic acid, beta-(o-chlorophenyl)propionic acid, beta-(m-chlorophenyl)propionic acid, beta-(p-chlorophenyl)propionic acid, beta-chloropropionic acid, cis-cinnamic acid, trans-cinnamic acid, o-cresol, m-cresol, p-cresol, trans-crotonic acid, cyclohexane-1:1-dicarboxylic acid, cyclopropane-1:1-dicarboxylic acid, DL-cysteine, L-cysteine, deuteroacetic acid, 2,3-dichlorophenol, 3,4-dihydroxybenzoic acid, 3,5-dihydroxybenzoic acid, dimethylglycine, dimethylmalic acid, 2,4-dintirophenol, 3,6-dinitrophenol, diphenylacetic acid, ethylbenzoic acid, formic acid, trans-fumaric acid, gallic acid, glutaramic acid, glutaric acid, glycine, glycolic acid, heptanoic acid, hexahydrobenzoic acid, hexanoic acid, hippuric acid, histidine, hydroquinone, m-hydroxybenzoic acid, p-hyroxybenzoic acid, beta-hyroxybutyric acid, gamma-hydroxybutyric acid, beta-hydroxypropionic acid, gamma-hydroxyquinoline, iodoacetic acid, m-iodobenzoic acid, itaconic acid, lysine, maleic acid, malic acid, malonic acid, DL-mandelic acid, mesaconic acid, mesitylenic acid, methyl-o-aminobenzoic acid, methyl-m-aminobenzoic acid, methyl-p-aminobenzoic acid, o-methylcinnamic acid, m-methylcinnamic acid, p-methylcinnamic acid, beta-methylglutaric acid, n-methylglycine, methylsuccinic acid, o-monochlorophenol, m-monochlorophenol, p-monochlorophenol, alpha-naphthoic acid, beta-naphthoic acid, alpha-naphthol, beta-naphthol, nitrobenzene, m-nitrobenzoic acid, p-nitrobenzoic acid, o-nitrophenol, m-nitrophenol, p-nitrophenol, o-nitrophenylacetic acid, m-nitrophenylacetic acid, p-nitrophenylacetic acid, o-beta-nitrophenylpropionic acid, m-beta-nitrophenylpropionic acid, p-beta-nitrophenylpropionic acid, nonanic acid, octanoic acid, oxalic acid, phenol, phenylacetic acid, o-phenylbenzoic acid, gamma-phenylbutyric acid, alpha-phenylpropionic acid, beta-phenylpropionic acid, o-phthalic, m-phthalic, p-phthalic, pimelic acid, propionic acid, iso-propylbenzoic acid, 2-pyridinecarboxylic acid, 3-pyridinecarboxylic acid, 4-pyridinecarboxylic acid, pyrocatecchol, resorcinol, saccharin, suberic acid, succinic acid, alpha-tartaric acid, meso-tartaric acid, theobromine, terephthalic acid, thioacetic acid, thiophenecarboxylic acid, o-toluic acid, m-toluic acid, p-toluic acid, trichlorophenol, trimethylacetic acid, tryptophan, tyrosine, uric acid, n-valeric, iso-valeric, veronal acid, vinylacetic acid, xanthine, salts thereof, and mixtures thereof.

Further suitable buffering agents can be, for example, arsenic acid, arsenious acid, o-boric acid, carbonic acid, chromic acid, germanic acid, hyrocyanic acid, hydrofluoric acid, hydrogen sulfide, hypobromous acid, nitrous acid, o-phosphoric acid, phosphorous acid, pyrophosphoric acid, selenious acid, m-silicic acid, o-silicic acid, sulfurous acid, telluric acid, tellureous acid, tetraboric acid, salts thereof, and mixtures thereof.

Buffering agents in the present compositions can be, for example, 3-chloropropanoic acid, citric acid, ethylenedinitrilotetraacetic acid (i.e., “EDTA”), alanine, aminobenzene, sulfanilic acid, 2-aminobenzoic acid, 2-aminophenol, ammonia, arginine, asparagine, aspartic acid, dimethyleneimine, benzene-1,2,3-tricarboxylic acid, benzoic acid, benzylamine, 2,2-bipyridine, butanoic acid, maleic acid, carbonic acid, dichloroacetic acid, diethylamine, catechol, resorcinol, d-tartaric acid, ethylenediamine, glutamic acid, glutamine, glycine, adipic acid, hydrogen hypophosphite, isoleucine, leucine, methionine, 3-nitrobenzoic acid, 4-nitrobenzoic acid, phthalic acid, iodoacetic acid, histidine, lysine, 4-methylaniline, ocresol, 2-naphthoic acid, nitrilotriacetic acid, 2-nitrobenzoic acid, 4-nitrophenol, 2,4-dinitrophenol, N-nitrosophenylhydroxylamine, nitrous acid, phosphoric acid, phenylalanine, piperdine, serine, hydrogen sulfite, threonine, tris(hydroxymethyl)aminomethane (i.e. “TRIS” or “THAM”), tyrosine; alkali metal salts thereof; and mixtures thereof. Still further suitable buffering agents can be, for example, zwitterionic buffers, such as MES, lysine, bisine, and like compounds, salts thereof, or mixtures thereof. Still further suitable buffering agents can be, for example, the so-called “Goode” buffers generally and which buffers may encompass some of the abovementioned buffers or compounds and which buffers are typically and advantageously biologically inert and do not interfere with biochemical reactions.

Surfactants

The buffer compositions of the present disclosure can optionally include one or more surfactant or a co-surfactant. The surfactant or co-surfactant can be nonionic surfactants, anionic surfactants, zwitterionic surfactants such as lauryl sarcosine, fluorocarbon surfactants (which are differentiated from the fluorocarbon superwetting agents of the present disclosure by, for example, having other structures, other surface activities, or both), and like surfactants, or mixtures thereof. An excellent source listing of surfactant materials is provided by McCutcheon's Vol. 1: Emulsifiers and Detergents, North American Ed., McCutheon Division, MC Publishing Co., 1995, the disclosure of which is incorporated herein by reference. Suitable nonionic surfactants include, but are not limited to, alkyl ethoxylated surfactants, block copolymer surfactants, castor oil surfactants, sorbitan ester surfactants, polyethoxylated fatty alcohol surfactants, glycerol mono-fatty acid ester surfactants, polyethylene glycol fatty acid ester surfactants, and mixtures thereof. Other useful nonionic alkyl alkoxylated surfactants are ethoxylated alkyl amines derived from the condensation of ethylene oxide with hydrophobic alkyl amines. Other examples of useful ethoxylated surfactants include carboxylated alcohol ethoxylate, also known as ether carboxylate.

Anionic surfactants can optionally be incorporated in the present compositions as a surfactant or co-surfactant. Many suitable non-limiting examples from the class of anionic surfactants can be found in McCutcheon's (supra) as well as Surfactants and Interfacial Phenomena, 2nd Ed., Milton J. Rosen, 1989, John Wiley & Sons, Inc., pp. 7-16, which is hereby incorporated by reference. Additional suitable non-limiting examples of anionic surfactants can be found in Handbook of Surfactants, M. R. Porter, 1991, Blackie & Son Ltd, pp. 54-115 and references therein, the disclosure of which is incorporated herein by reference.

Hybridization

As mentioned above, in general, nucleic acid hybridizations can comprise prehybridization treatment to increase accessibility of target DNA, and to reduce nonspecific binding; hybridization of the mixture of nucleic acid sequences to the nucleic acid on the solid surface; posthybridization washes to remove nucleic acid fragments not bound in the hybridization and detection of the hybridized nucleic acid fragments.

There are numerous types of substrates used in hybridization assays. Common substrates or supports used for array assays are surface-modified siliceous substrates, such as glass. DNA microarrays are typically, but not always, synthesized or deposited onto these substrates. The substrate surface can be modified to enable or facilitate the initial attachment of nucleic acids to the surface for the manufacture of the array probes.

Surface modification or derivatization techniques are known in the art. A common surface derivatization is silane-based.

Arrays of oligomer probes, such as oligonucleotides or polynucleotides, can be hybridized using conventional methods and hybridization solutions. J. Sambrook, E. F.

Fritsch, T. Maniatis, Molecular Cloning: A Laboratory Manual, Cold Spring Harbor Laboratory Press, Cold Spring Harbor, 2nd Ed., 1989, vol. 1-3, incorporated herein by reference, describe the considerations and conditions for hybridization of oligonucleotide probes. Probe length, hybridization temperature, as well as other factors well known in the art affect hybridization conditions. Typically, hybridizations using synthetic oligomers are usually carried out under conditions that are 5-10° C. below the calculated melting temperature Tₘ of a perfect hybrid to minimize mismatched or non-Watson/Crick base pairing between the probe and target, and maximize the rate at which Watson/Crick base pairs form. Other factors influencing the rate of hybrid formation include the salt concentration, the presence of solvents or co-solvents, the concentration of nucleic acid in solution, the length of hybridization, and the degree and method of agitation.

A conventional hybridization solution can comprise, for example, a salt (e.g. a monovalent cation), a buffer that provides buffering capacity between pH 6.8-8.5 (more typically between pH 7.0-7.5), a divalent cation chelating agent (e.g., ethylenediaminetetraacetic acid, EDTA), and agents for blocking non-specific binding of targets to the array surface, such as surfactants, proteins, a carrier DNA from an organism unrelated to the experiment at hand, and like ingredients. A typical hybridization solution contains 6× SSPE (0.9 M NaCl, 60 mM sodium phosphate (pH 7.4); 6 mM EDTA); or 6×SSC (0.9 M NaCl, 90 mM sodium citrate (pH 7.0)), 0.5% w/v sodium dodecyl sulfate (SDS); 100 micrograms/mL denatured, fragmented salmon sperm DNA; and 0.1% w/v nonfat dried milk. In some embodiments, a hybridization solution can comprise a superwetting agent as disclosed herein.

An array can be hybridized according to standard protocols as disclosed or referenced herein for a period of time ranging from about 2 hours to about 2 days, depending on at least the make-up of the probes (i.e., probe length and diversity of probe composition) and the complexity of the target, for example, at a controlled temperature, which typically ranges from 20° C. to 70° C., depending on the melting temperature Tₘ, as discussed above. Low temperature hybridizations are performed at about 20° C. to about 50° C. (typically about 37-45° C.). High temperature hybridizations are performed at or around 55° C. to about 70° C. (typically 60° C. to 65° C.). However, for most nucleic acid microarrays, high temperature hybridizations are preferred in the art since the higher temperature maximizes the rate of Watson/Crick base pairing of nucleotides. The typical time period for hybridization of an array is overnight or longer (i.e., anywhere from 8 hours to at least 24 hours) so as to hybridize the target. The array is then washed (with various wash and soak steps as described herein), dried and optically scanned to measure the degree of hybridization.

In high-throughput applications, hybridization chambers can be used. A “chamber” references an enclosed volume (although a chamber may be accessible through one or more ports). A microarray hybridization chamber can have, for example, a thickness of about 0.4 mm, and contain about 0.5 mL of hybridization solution. Exemplary chambers are described in U.S. Pat. Nos. 7,115,423; 7,125,523; 6,946,287; and 6,258,593 and U.S. Pat. Publication Nos. 20060003440 and 20060234267 and are available commercially (e.g., from Agilent (e.g., part no. G2534A), Affymetrix (GeneChip®), and Tecan (e.g., part no. HS4800)).

A number of different protocols have been reported for effecting mixing during hybridization of DNA microarrays. These include, for example, the procedures described for the use of Agilent microarrays, the Affymetrix GeneChip®, the Amersham Codelink® Biochip, and the BioMicro MAUI® Hybridization System. The methods and compositions of the present disclosure can provide improved post-hybridization processing techniques with any of these protocols.

The methods and composition of the present disclosure need not limited to uses in DNA hybridization. Systems that are designed for the analysis of proteins or small molecules, such as protein or peptide arrays, encounter many of the same post-binding problems as in DNA hybridization, and can benefit from the use of superwetting agents of the present disclosure. Other systems, such as microfluidic based assays, in which it is desirable for fluids to be transported through narrow channels or capillaries, can also benefit from the use of superwetting agents.

### EXAMPLES

The following examples serve to more fully describe the manner of using the above-described disclosure, as well as to set forth the best modes contemplated for carrying out various aspects of the disclosure. It is understood that these examples in no way serve to limit the true scope of this disclosure, but rather are presented for illustrative purposes.

### Example 1

### Preparation of a Wash Buffer Containing a Superwetting Agent

A Wash Buffer composition containing a superwetting agent was prepared as follows:

TEGO® Wet 260 was spiked into a buffer comprising 0.06× SSPE, 0.005% (w/v) n-lauroyl sarcosine. The superwetting agent was spiked at the ratio of 100 μL per 1 liter for a final concentration of 0.01% (v/v).

### Example 2

### Monitoring Gene Expression

Generally, methods of monitoring gene expression can involve providing a pool of target nucleic acids comprising RNA transcript(s) of one or more target genes(s), or nucleic acids derived from the RNA transcript(s); hybridizing the nucleic acid sample to a high density array of probes; and detecting the hybridized nucleic acids and calculating a relative expression level.

The present example utilized Agilent's Low RNA Input Linear Amplification Kit PLUS which generates fluorescent cRNA (complimentary RNA) with a sample input RNA range between 50 ng and 5 μg of total RNA or a minimum of 10 ng of poly A+ RNA for two-color processing. The method uses T7 RNA polymerase, which simultaneously amplifies target material and incorporates cyanine 3- or cyanine 5-labeled CTP. There is routinely at least a 100-fold RNA amplification with use of this kit.

cRNA was derived from 2 cell lines: Leukemia (K-562) Total RNA (Ambion, Catalog no. 7832) and Osteosarcoma (MG-63) Total RNA (Ambion, Catalog no. 7868). The K-562 cRNA (labeled using cyanine 5-CTP) and the MG-63 cRNA (labeled using cyanine 3-CTP) were pooled and used as the hybridization target sample.

Hybridization of the target sample to an Agilent 44 K feature array (Agilent catalog no. G4410B) was performed using a Tecan HS 400 Pro Hybridization Station (Tecan, Research Triangle Park, N.C., USA).

The reagent bottle set-up is shown in the following table:

| Wash Bottle | Reagent | Heated |
|---|---|---|
| 1 | Prehyb Buffer | Yes |
| 2 | Gene Expression Wash | No |
| | Buffer 1 | |
| 3 | Gene Expression Wash | Yes |
| | Buffer 2* | |
| 4 | Blank | N/A |
| 5 | Water | No |
| 6 | Cleaning and Conditioning | No |
| | Solution | |

*When present, TEGO ® Wet 260 was spiked into the Gene Expression Wash Buffer 2 (Agilent catalog no. 5188–5222) at the ratio of 100 uL per 1 liter of wash buffer.

The hybridization volumes are shown in the following table:

| | Volume (μL) per hybridization |
|---|---|
| Component | (44K microarray) |
| Cyanine 5- and cyanine 3-labeled | 57.6 |
| cRNA mixture | |
| 25X Fragmentation buffer | 2.4 |
| (Agilent catalog no. 5185–5974) | |
| 10X Blocking Agent | 5 |
| (Agilent catalog no. 5188–5281) | |
| 2X Hybridization Buffer | 65 |
| (Agilent catalog no. 5185–5973) | |
| Final Hybridization Sample | 130 |
| Volume | |

The Tecan HS 400 Pro was programmed according to the following protocol:

| Step Number | Program Step | Step Action |
|---|---|---|
| 1 | Wash | Pre-hybridization Buffer at |
| | | 67° C. |
| 2 | Sample Injection | Sample Loading (120 μL) |
| 3 | Hybridization | 67° C. for 24 hours |
| 4 | Wash | Gene Expression Wash Buffer 1 |
| | | at Room Temp* |
| 5 | Wash | Gene Expression Wash Buffer 2 |
| | | at 37° C.** |
| 6 | Drying | 2 minutes at 30° C. |

*Gene Expression Wash Buffer 1: 0.6x SSPE, 0.005% n-lauroyl sarcosine.
**Gene Expression Wash Buffer 2: 0.06x SSPE, 0.005% n-lauroyl sarcosine.

Step 4 wash (repeated 2 times) included a 1 min wash, followed by a 1 min soak. Step 5 wash (repeated 2 times) included a 1 min wash, followed by a 1 min soak. The step 6 drying was under N₂ at 39 psi.

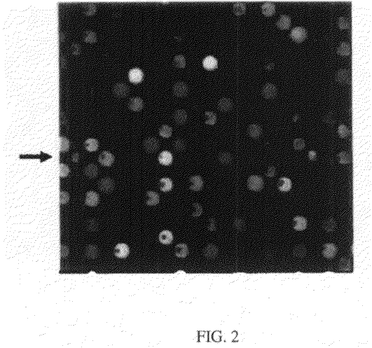

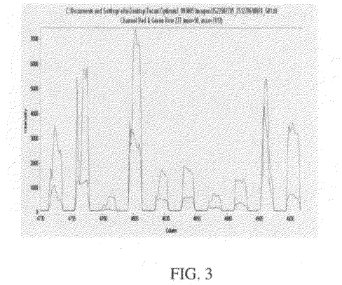

Referring to the Figures, FIG. 2 shows features on a 44 K feature gene expression microarray after washing with standard Wash Buffer 2 (0.06× SSPE, 0.005% sarcosine). The microarray was dried using 39 psi of N₂ gas. Dark regions on features are due to non-uniform feature drying. FIG. 3 shows a feature extraction line scan across a scan line (indicated by the arrow in FIG. 2) and demonstrates non-uniformity within features.

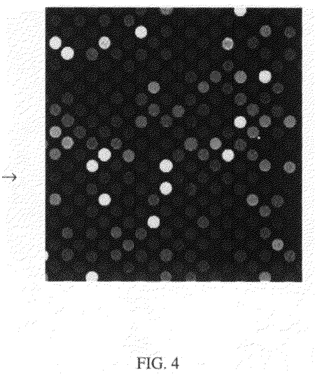

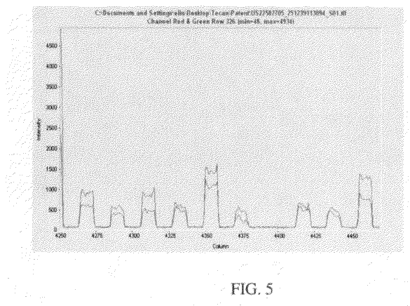

FIG. 4 shows features on a 44 K feature gene expression microarray after washing with a Wash Buffer 2 that included TEGO® Wet 260 (0.06× SSPE, 0.005% sarcosine, 0.01% (v/v) TEGO® Wet 260), followed by drying under N₂ gas. FIG. 5 shows a feature extraction line scan across a scan line (arrow in FIG. 4) and demonstrates substantial uniformity within features. As shown in FIGS. 4 and 5, use of Wash Buffer 2 containing TEGO Wet 260 significantly increased the uniformity within features as compared to the use of the Wash Buffer 2 lacking TEGO Wet 260 (FIGS. 2 and 3). In FIGS. 2-5, the cytidine-5 signal is shown in red, the cytidine-3 label is shown in green, and yellow indicates approximately equal amounts of cytidine-3 and cytidine-5.

### Example 3

### Effect of Superwetting Agent in a Wash Buffer on the Distribution of Non-Uniform Features

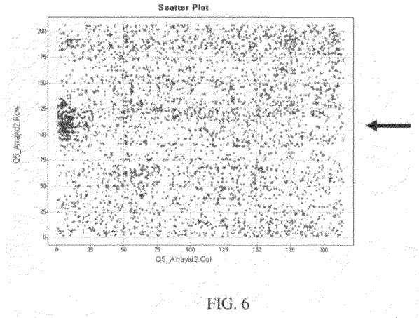

FIG. 6 shows the spatial distribution of features flagged as non-uniform from a microarray processed as described in Example 2 and using standard Wash Buffer 2 (i.e., without TEGO Wet 260). The x-axis represents the microarray column and the y-axis represents the microarray row. The arrow on the right side represents the direction from which N₂ gas was delivered for microarray drying. The cluster of non-uniform features on the left side of the microarray is around the hybridization chamber outlet port (not shown).

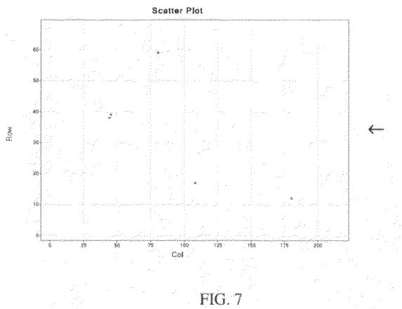

FIG. 7 shows the spatial distribution of features flagged as non-uniform from a microarray processed using Wash Buffer 2 containing TEGO Wet 260 (0.01%). As shown, the use of the superwetting agent in the wash buffer greatly reduced the clustering of non-uniform features. With the addition of 0.01% TEGO Wet 260 to Wash Buffer 2, the number of features flagged as non-uniform dropped from greater than 5% to less than 1%.

### Example 4

### Effect of a Superwetting Agent in a Wash Buffer on the Distribution of Non-Uniform Background Areas

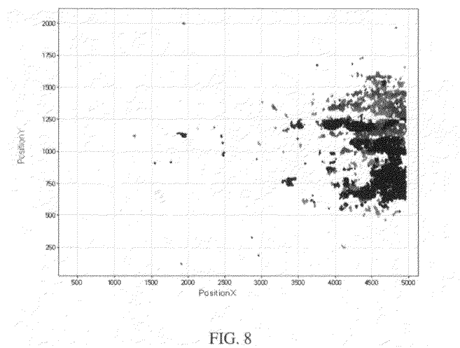

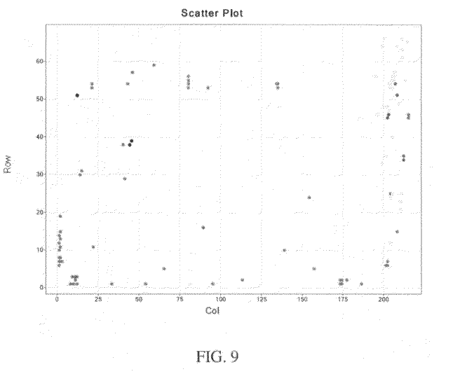

The following shows the spatial distribution of background areas that were observed to be non-uniform on a microarray processed as described in Example 2 (with a drying pressure of 20 psi of N₂ gas). In FIG. 8, a standard Wash Buffer 2 (without TEGO Wet 260) was used. The data show the pattern of locations for the background population outliers from four individual microarrays, indicated by different colors, respectively. The x-axis represents the microarray column and the y-axis represent the microarray row. The clustering of non-uniform background areas on the right side of each microarray is near the hybridization chamber inlet port (not shown). In FIG. 9, Wash Buffer 2 containing TEGO Wet 260 (0.01%) was used and resulted in a significant reduction in the clustering of non-uniform background areas.

The disclosure has been described with reference to various specific embodiments and techniques. Additional aspects of the disclosure are additionally described and illustrated in the Figure(s) provided. However, it should be understood that many variations and modifications are possible while remaining within the spirit and scope of the disclosure.

What is claimed is:

1. A method of treating a hybrid formed from a first oligonucleotide bound to a surface with a second unbound oligonucleotide, the method comprising: subjecting the surface to processing with an aqueous mixture comprising a superwetting agent after formation of the hybrid.

2. The method according to claim 1 wherein the superwetting agent comprises an organosilicone wetting agent, an organofluorine wetting agent, a hydrocarbyl wetting agent, or mixtures thereof.

3. The method according to claim 1 wherein the superwetting agent comprises a polyether siloxane copolymer.

4. The method according to claim 1 wherein the buffer capacity and the pH of the aqueous mixture is at from about 5 to about 9.

5. The method according to claim 1 wherein the contacting is accomplished with a moving or collapsing gas bubble.

6. The method according to claim 1 wherein the surface is a silane surface-modified siliceous microarray.

7. The method of claim 1 wherein the post-hybridization processing comprises at least one washing step.

8. The method of claim 1 wherein the post-hybridization processing comprises a soaking step after said washing step.

9. The method of claim 1 wherein the post-hybridization processing comprises a plurality of washing steps.

10. The method of claim 1 wherein the post-hybridization processing comprises a drying step after said washing step.

11. The method of claim 1 wherein said surface is exposed to said superwetting agent in a washing step immediately preceding a drying step.

12. A method of processing a ligand array comprising one or more ligands immobilized on a surface of a solid support, said method comprising:
contacting said surface with the wash buffer of claim 1.

13. The method of claim 12, wherein said contacting step is followed by separating said wash buffer from said surface.

14. A wash buffer comprising: a superwetting agent and a buffer suitable for use in a microarray post-hybridization washing procedure.

15. The wash buffer of claim 14 wherein the buffer comprises an organic compound, an inorganic compound, or mixtures thereof.

16. The wash buffer of claim 14 wherein: the superwetting agent is present from about 0.0001 to about 2 weight percent.

17. The wash buffer of claim 14 capable of drying uniformly.

18. A wash buffer comprising a siloxane surfactant.

19. The wash buffer of claim 18, wherein said siloxane surfactant comprises a polyether siloxane.

20. The wash buffer of claim 19, wherein said polyether siloxane comprises a polyether siloxane copolymer.

21. The wash buffer of claim 18, comprising about 15 mM to about 6 M of a salt.

22. The wash buffer of claim 21, wherein said salt is NaCl.

23. The wash buffer of claim 18, comprising about 1 mM to about 0.6 M of a buffer salt.

24. The wash buffer of claim 23, wherein said buffer salt is sodium citrate or sodium phosphate.

25. The wash buffer of claim 18, comprising about 0.1 mM to about 0.04 M of a metal chelator.

26. The wash buffer of claim 25, wherein said metal chelator is EDTA.

27. The wash buffer of claim 18, comprising an amino acid.

28. The wash buffer of claim 27, wherein said amino acid is sarcosine.

29. The wash buffer of claim 18, comprising about 0.0001% to about 2.0% of said siloxane surfactant.

30. The wash buffer of claim 18, comprising about 0.001% to about 0.1% of said siloxane surfactant.

31. The wash buffer of claim 18, comprising about 0.005% to about 0.02% of said siloxane surfactant.

32. The wash buffer of claim 18 suitable for separating a first nucleic acid from a second nucleic acid immobilized on a surface of a solid support.

33. The wash buffer of claim 18 capable of drying uniformly.

34. A method of processing a ligand array comprising one or more ligands immobilized on a surface of a solid support, said method comprising:
contacting said surface with the wash buffer of claim 18.

35. The method of claim 34, wherein said contacting step is followed by separating said wash buffer from said surface.

36. The method of claim 35, wherein said separating comprises evaporating of said wash buffer.

37. A kit comprising:
(a) said wash buffer of claim 18, and
(b) a ligand array comprising one or more ligands immobilized on a surface of a solid support.

38. A kit for use in post-hybridization processing of a hybrid formed between an oligonucleotide material and oligonucleotides attached to the surface in a microarray, the kit comprising:
a buffer comprising a superwetting agent; a microarray having a plurality of oligonucleotides attached to the surface in an array pattern of features; and instructions for using the buffer concentrate in post-hybridization processing of hybrids formed on the microarray.

39. A system for high-throughput microarray processing, the system comprising: a wash buffer comprising a superwetting agent.

40. The system of claim 39 comprising a chamber for holding a microarray.
